(19) United States
(12) Patent Application Publication
(10) Pub. No.: US 2024/0045086 A1
(43) Pub. Date: Feb. 8, 2024

(54) IMAGING METHOD USING SEMICONDUCTOR RADIATION DETECTOR

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 18/382,658

(22) Filed: Oct. 23, 2023

Related U.S. Application Data
(63) Continuation of application No. PCT/CN2021/091867, filed on May 6, 2021.

(51) Int. Cl. G01T 1/24; A61B 6/00

(52) U.S. Cl. CPC G01T 1/243; A61B 6/504

(57) ABSTRACT

Disclosed herein is a method comprising emitting particles of radiation from a first position on a radiation source toward a scene; capturing a first partial image of the scene by an image sensor using the particles of radiation from only the first position; emitting particles of radiation from a second position on the radiation source toward the scene, the second position being different from the first position relative to the scene; capturing a second partial image of the scene by the image sensor using the particles of radiation from only the second position; forming an image of the scene by stitching the partial images; wherein the image sensor has dead zones among radiation detectors arranged in strips; wherein a portion of the scene in the first partial image is formed by the particles of radiation from only the first position falls on the dead zones of the image sensor.

### BACKGROUND

Radiation detectors may be devices used to measure the flux, spatial distribution, spectrum or other properties of radiations.

Radiation detectors may be used for many applications. One important application is imaging. Radiation imaging is a radiography technique and can be used to reveal the internal structure of a non-uniformly composed and opaque object such as the human body.

Early radiation detectors for imaging include photographic plates and photographic films. A photographic plate may be a glass plate with a coating of light-sensitive emulsion. Although photographic plates were replaced by photographic films, they may still be used in special situations due to the superior quality they offer and their extreme stability. A photographic film may be a plastic film (e.g., a strip or sheet) with a coating of light-sensitive emulsion.

In the 1980s, photostimulable phosphor plates (PSP plates) became available. A PSP plate may contain a phosphor material with color centers in its lattice. When the PSP plate is exposed to radiation, electrons excited by radiation are trapped in the color centers until they are stimulated by a laser beam scanning over the plate surface. As the plate is scanned by laser, trapped excited electrons give off light, which is collected by a photomultiplier tube. The collected light is converted into a digital image. In contrast to photographic plates and photographic films, PSP plates can be reused.

Another kinds of radiation detectors are radiation image intensifiers. Components of a radiation image intensifier are usually sealed in a vacuum. In contrast to photographic plates, photographic films, and PSP plates, radiation image intensifiers may produce real-time images, i.e., do not require post-exposure processing to produce images. Radiation first hits an input phosphor (e.g., cesium iodide) and is converted to visible light. The visible light then hits a photocathode (e.g., a thin metal layer containing cesium and antimony compounds) and causes emission of electrons. The number of emitted electrons is proportional to the intensity of the incident Radiation. The emitted electrons are projected, through electron optics, onto an output phosphor and cause the output phosphor to produce a visible-light image.

Scintillators operate somewhat similarly to radiation image intensifiers in that scintillators (e.g., sodium iodide) absorb radiation and emit visible light, which can then be detected by a suitable image sensor for visible light. In scintillators, the visible light spreads and scatters in all directions and thus reduces spatial resolution. Reducing the scintillator thickness helps to improve the spatial resolution but also reduces absorption of radiation. A scintillator thus has to strike a compromise between absorption efficiency and resolution.

Semiconductor radiation detectors largely overcome this problem by direct conversion of radiation into electric signals. A semiconductor radiation detector may include a semiconductor layer that absorbs radiation in wavelengths of interest. When a particle of radiation is absorbed in the semiconductor layer, multiple charge carriers (e.g., electrons and holes) are generated and swept under an electric field towards electric contacts on the semiconductor layer. Cumbersome heat management required in currently available semiconductor radiation detectors (e.g., Medipix) can make a detector with a large area and a large number of pixels difficult or impossible to produce.

### SUMMARY

Disclosed herein is a method comprising: emitting particles of radiation from a first position on a radiation source toward a scene; capturing a first partial image of the scene by an image sensor using the particles of radiation from only the first position; emitting particles of radiation from a second position on the radiation source toward the scene, the second position being different from the first position relative to the scene; capturing a second partial image of the scene by the image sensor using the particles of radiation from only the second position; forming an image of the scene by stitching the partial images; wherein the image sensor comprises radiation detectors arranged in strips; wherein the image sensor has dead zones among the strips; wherein a portion of the scene in the first partial image is formed by the particles of radiation from only the first position falls on the dead zones of the image sensor; wherein the portion of the scene in the second partial image is formed by the particles of radiation from only the second position falls on active areas of the image sensor.

According to an embodiment, the image sensor remains stationary relative to the scene.

According to an embodiment, each point in the scene is captured in at least two partial images formed by particle of radiation from different positions on the radiation source.

According to an embodiment, the radiation source is stationary relative to the scene.

According to an embodiment, the radiation source comprises an electron gun and an electron bombardment target.

According to an embodiment, the radiation source is configured to cause electrons from the electron gun to bombard the electron bombardment target at the first position or the second position.

According to an embodiment, the radiation source is configured to cause electrons from the electron gun to bombard the electron bombardment target at the first position or the second position by moving the electron bombardment target relative to the electron gun.

According to an embodiment, the electron bombardment target is configured to tilt, translate, or both tilt and translate.

According to an embodiment, the electron gun is configured to generate an electron beam and then deflect the electron beam.

According to an embodiment, the electron bombardment target comprises tungsten.

According to an embodiment, the image sensor comprises a plurality of radiation detectors wherein the radiation detectors are configured to count numbers of particles of radiation incident on the detectors, within a period of time.

According to an embodiment, the particles of radiation are X-ray photons.

According to an embodiment, at least one of the radiation detectors comprises: a radiation absorption layer comprising an electric contact; a first voltage comparator configured to compare a voltage of the electric contact to a first threshold; a second voltage comparator configured to compare the voltage to a second threshold; a counter configured to register a number of particles of radiation incident on the radiation absorption layer; a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause at least one of the numbers of particles to increase by one, when the second voltage comparator determines that an absolute value of the voltage equals or exceeds an absolute value of the second threshold.

According to an embodiment, the image sensor further comprises an integrator electrically connected to the electric contact, wherein the integrator is configured to collect charge carriers from the electric contact.

According to an embodiment, the controller is configured to activate the second voltage comparator at a beginning or expiration of the time delay.

According to an embodiment, the controller is configured to connect the electric contact to an electrical ground.

According to an embodiment, a rate of change of the voltage is substantially zero at expiration of the time delay.

According to an embodiment, the radiation absorption layer comprises a diode.

According to an embodiment, the radiation absorption layer comprises single-crystalline silicon.

According to an embodiment, the radiation detector does not comprise a scintillator.

Disclosed here is a digital subtraction angiography imaging system implementing a method of any one of above mentioned.

### BRIEF DESCRIPTION OF FIGURES

FIG. 1 schematically shows a top view of a portion of a radiation detector 100, according to an embodiment.

FIG. 2A schematically shows a cross-sectional view of the radiation detector 100, according to an embodiment.

FIG. 2B schematically shows a detailed cross-sectional view of the radiation detector 100, according to an embodiment.

FIG. 2C schematically shows an alternative detailed cross-sectional view of the radiation detector 100, according to an embodiment.

FIG. 3A schematically shows a top view of a package 200 including the radiation detector 100 and a printed circuit board (PCB) 400, according to an embodiment.

FIG. 3B schematically shows a cross-sectional view of an image sensor 9000, according to an embodiment.

FIG. 4A and FIG. 4B schematically show perspective views of an imaging system suitable for digital subtraction angiography, according to an embodiment.

FIG. 5 schematically shows the image sensor 9000 capturing a plurality of partial images of portions of the scene 50, according to an embodiment.

FIG. 6 schematically shows a flowchart for a method of operating the imaging system of FIGS. 4A and 4B, according to an embodiment.

FIG. 7A and FIG. 7B each show a component diagram of an electronic system of the radiation detector in FIG. 2A, FIG. 2B and FIG. 2C, according to an embodiment.

FIG. 8 schematically shows a temporal change of the electric current flowing through an electrode (upper curve) of a diode or an electric contact of a resistor of a radiation absorption layer exposed to radiation, the electric current caused by charge carriers generated by a particle of radiation incident on the radiation absorption layer, and a corresponding temporal change of the voltage of the electrode (lower curve), according to an embodiment.

### DETAILED DESCRIPTION

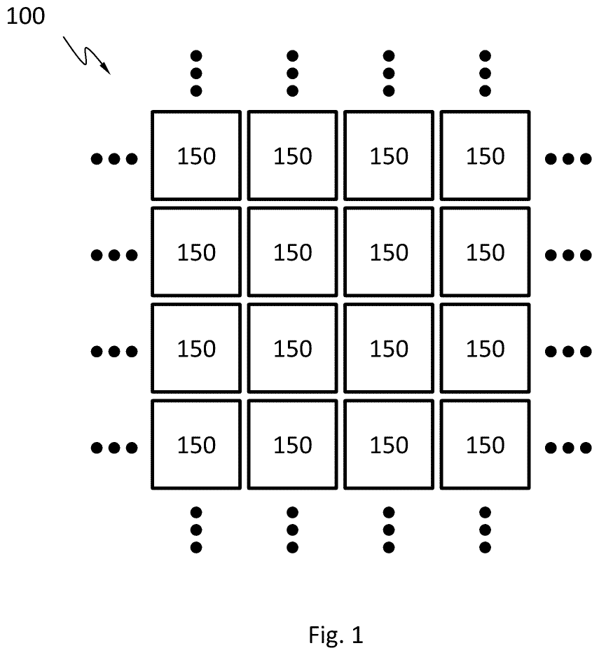

FIG. 1 schematically shows a top view of a portion of a radiation detector 100, according to an embodiment. The radiation detector 100 may have an array of pixels 150, according to an embodiment. The array may be a rectangular array, a honeycomb array, a hexagonal array or any other suitable array. Each pixel 150 may be configured to detect particles of radiation incident thereon, to measure the energy of the particle of radiation, or both. The particles of radiation may be X-ray photons. For example, each pixel 150 may be configured to count numbers of particles of radiation incident thereon whose energy falls in a plurality of bins, within a period of time. All the pixels 150 may be configured to count the numbers of particles of radiation incident thereon within a plurality of bins of energy within the same period of time. Each pixel 150 may have its own analog-to-digital converter (ADC) configured to digitize an analog signal representing the energy of an incident particle of radiation into a digital signal. The ADC may have a resolution of 10 bits or higher. Each pixel 150 may be configured to measure its dark current, such as before or concurrently with each particle of radiation incident thereon. Each pixel 150 may be configured to deduct the contribution of the dark current from the energy of the particle of radiation incident thereon. The pixels 150 may be configured to operate in parallel. For example, when one pixel 150 measures an incident particle of radiation, another pixel 150 may be waiting for another particle of radiation to arrive. The pixels 150 may be but do not have to be individually addressable.

The radiation detector 100 described here may have applications such as in X-ray digital subtraction angiography, X-ray mammography, industrial X-ray defect detection, X-ray microscopy or microradiography, X-ray casting inspection, X-ray non-destructive testing, X-ray weld inspection, or an X-ray telescope, etc.

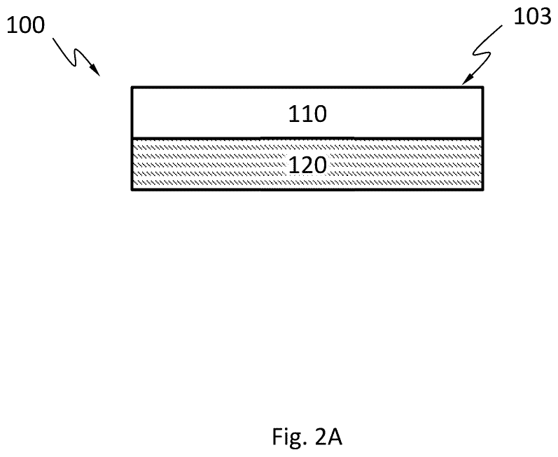

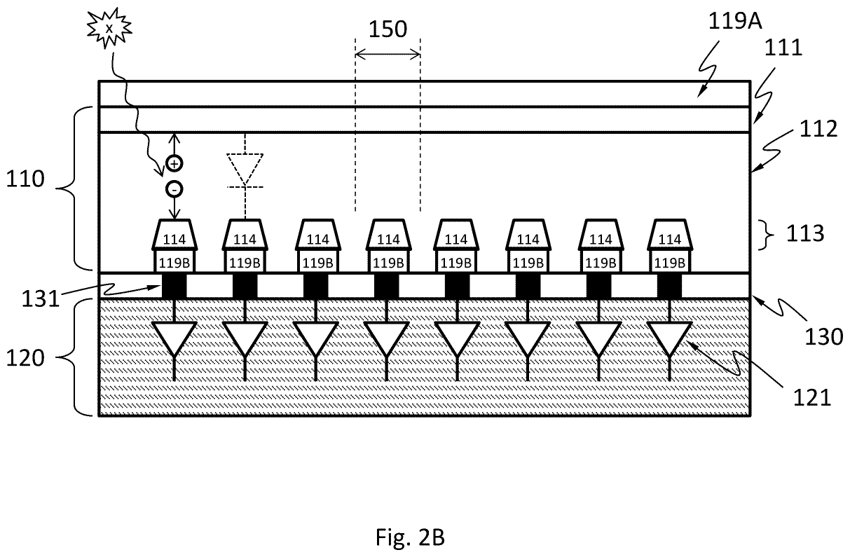

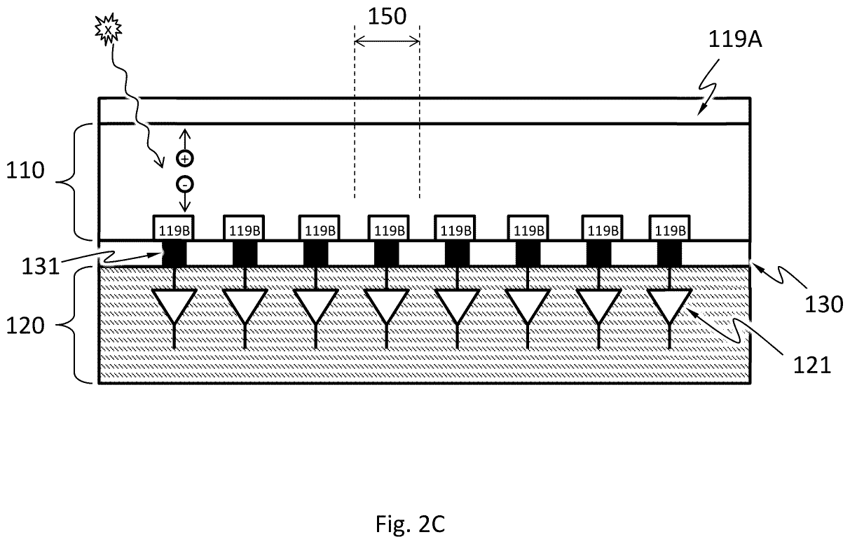

FIG. 2A schematically shows a cross-sectional view of the radiation detector 100, according to an embodiment. The radiation detector 100 may include a radiation absorption layer 110 and an electronics layer 120 (e.g., an ASIC) for processing or analyzing electrical signals incident radiation generates in the radiation absorption layer 110. The radiation detector 100 may not comprise a scintillator. The radiation absorption layer 110 may comprise a semiconductor material such as, silicon, germanium, GaAs, CdTe, CdZnTe, or single-crystalline silicon. The semiconductor may have a high mass attenuation coefficient for the radiation energy of interest. In one embodiment, the surface 103 of the radiation absorption layer 110 distal from the electronics layer 120 is configured to receive incident radiation.

As shown in a detailed cross-sectional view of the radiation detector 100 in FIG. 2B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes having the first doped region 111 as a shared electric contact. The first doped region 111 may also have discrete portions.

When a particle of radiation hits the radiation absorption layer 110 including diodes, the particle of radiation may be absorbed and generate one or more charge carriers by a number of mechanisms. A particle of radiation may generate 10 to 100000 charge carriers. The charge carriers may drift to the electric contacts of one of the diodes under an electric field. The field may be an external electric field. The electric contact 119B may include discrete portions each of which is in electrical contact with the discrete regions 114. In an embodiment, the charge carriers may drift in directions such that the charge carriers generated by a single particle of radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of radiation incident around the footprint of one of these discrete regions 114 are not substantially shared with another of these discrete regions 114. The pixel 150 associated with a discrete region 114 may be an area around the discrete region 114 in which substantially all (more than 98%, more than 99.5%, more than 99.9%, or more than 99.99% of) charge carriers generated by a particle of radiation incident therein at an angle of incidence of 0° flow to the discrete region 114. Namely, less than 2%, less than 1%, less than 0.1%, or less than 0.01% of these charge carriers flow beyond the pixel.

As shown in an alternative detailed cross-sectional view of the radiation detector 100 in FIG. 2C, according to an embodiment, the radiation absorption layer 110 may comprise a resistor of a semiconductor material such as, silicon, germanium, GaAs, CdTe, CdZnTe, or a combination thereof, but does not include a diode. The semiconductor may have a high mass attenuation coefficient for the radiation energy of interest.

When a particle of radiation hits the radiation absorption layer 110 comprising a resistor but not diodes, it may be absorbed and generate one or more charge carriers by a number of mechanisms. A particle of radiation may generate 10 to 100000 charge carriers. The charge carriers may drift to the electric contacts 119A and 119B under an electric field. The field may be an external electric field. The electric contact 119B includes discrete portions. In an embodiment, the charge carriers may drift in directions such that the charge carriers generated by a single particle of radiation are not substantially shared by two different discrete portions of the electric contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of radiation incident around the footprint of one of these discrete portions of the electric contact 119B are not substantially shared with another of these discrete portions of the electric contact 119B. The pixel 150 associated with a discrete portion of the electric contact 119B may be an area around the discrete portion in which substantially all (more than 98%, more than 99.5%, more than 99.9% or more than 99.99% of) charge carriers generated by a particle of radiation incident at an angle of incidence of 0° therein flow to the discrete portion of the electric contact 119B. Namely, less than 2%, less than 0.5%, less than 0.1%, or less than 0.01% of these charge carriers flow beyond the pixel associated with the one discrete portion of the electric contact 119B.

The electronics layer 120 may include an electronic system 121 suitable for processing or interpreting signals generated by particles of radiation incident on the Radiation absorption layer 110. The electronic system 121 may include an analog circuitry such as a filter network, amplifiers, integrators, and comparators, or a digital circuitry such as a microprocessor, and memory. The electronic system 121 may include components shared by the pixels or components dedicated to a single pixel. For example, the electronic system 121 may include an amplifier dedicated to each pixel and a microprocessor shared among all the pixels. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

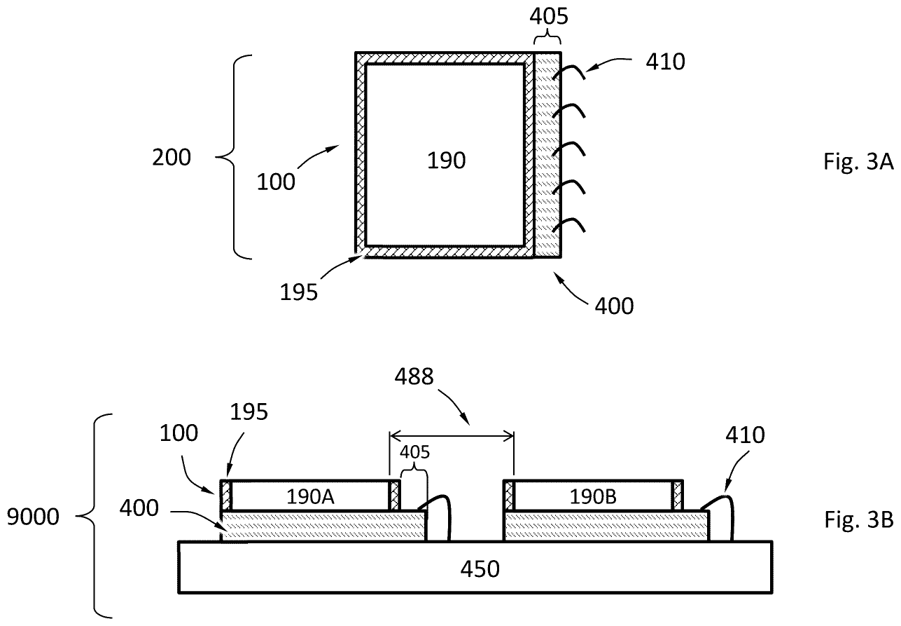

FIG. 3A schematically shows a top view of a package 200 including the radiation detector 100 and a printed circuit board (PCB) 400, according to an embodiment. The term “PCB” as used herein is not limited to a particular material. For example, a PCB may include a semiconductor. In one embodiment, the radiation detector 100 is mounted to the PCB 400. The PCB 400 may have one or more radiation detectors 100. The PCB 400 may have an area 405 not covered by the radiation detector 100 (e.g., for accommodating bonding wires 410). The radiation detector 100 may have an active area 190, which is where the pixels 150 (FIG. 1) are located. The radiation detector 100 may have a perimeter zone 195 near the edges of the radiation detector 100. The perimeter zone 195 may have no pixels and the radiation detector 100 may not detect particles of radiation incident on the perimeter zone 195.

FIG. 3B schematically shows a cross-sectional view of an image sensor 9000, according to an embodiment. The image sensor 9000 may include a plurality of the packages 200 of FIG. 3A mounted to a system PCB 450. The electrical connection between the PCBs 400 and the system PCB 450 may be made by bonding wires 410. To accommodate the bonding wires 410 on the PCB 400, the area 405 may be not covered by the radiation detector 100. To accommodate the bonding wires 410 on the system PCB 450, the packages 200 may have gaps in between. The gaps may be approximately 1 mm or more. Particles of radiation incident on the perimeter zones 195, on the area 405 or on the gaps may not be detected by the packages 200 on the system PCB 450. A dead zone of a radiation detector (e.g., the radiation detector 100) is the area of the radiation-receiving surface of the radiation detector, in which incident particles of radiation cannot be detected by the radiation detector. A dead zone of a package (e.g., package 200) is the area of the radiation-receiving surface of the package, in which incident particles of radiation cannot be detected by the detector or detectors in the package. In the examples shown in FIGS. 3A and 3B, the dead zone of the package 200 includes the perimeter zones 195 and the area 405. A dead zone (e.g., 488) of an image sensor (e.g., image sensor 9000) with a group of packages (e.g., packages mounted on the same PCB, packages arranged in the same layer) includes the combination of the dead zones of the packages in the group and the gaps among the packages.

The image sensor 9000 including the radiation detectors 100 may have the dead zone 488 incapable of detecting incident radiation. However, the image sensor 9000 may capture images of all points of an object (not shown), and then these captured images may be stitched to form a full image of the entire object.

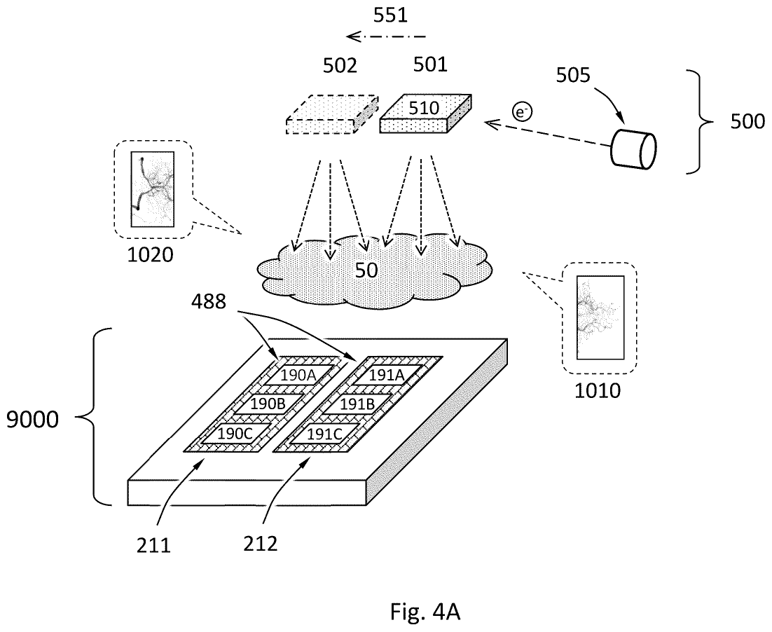

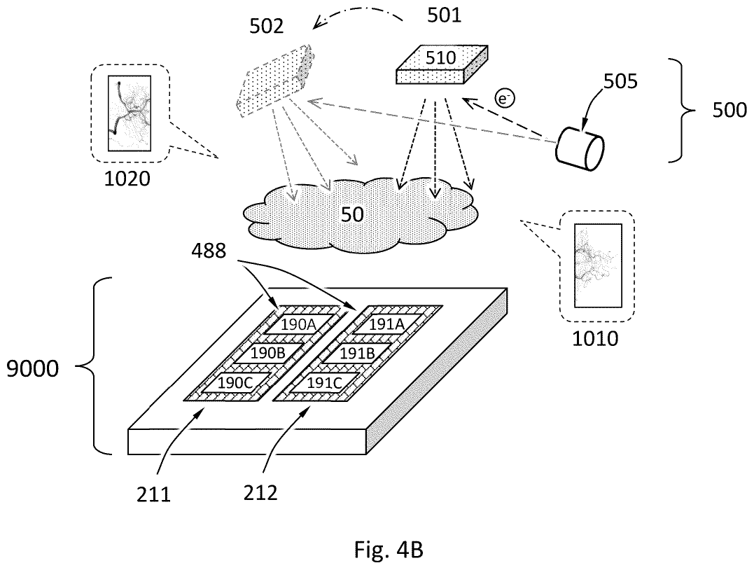

FIG. 4A and FIG. 4B schematically show perspective views of an imaging system comprising the image sensor 9000 and a radiation source 500, according to an embodiment. The imaging system may be used to perform digital subtraction angiography. As an example, the image sensor 9000 in FIG. 4A may comprise 6 radiation detectors 100 represented by their active areas 190A, 190B, 190C, and 191A, 191B, 191C (or collectively 190A-C and 191A-C for simplicity) which may be grouped into two strips 211 and 212. In one embodiment, the dead zone 488 is surrounding the 6 active areas 190A-C, 191A-C, and among the strips 211, 212, which is incapable of detecting incident particles of radiation. The radiation source 500 may comprise an electron gun 505, an electron bombardment target 510. The radiation source 500 is configured to cause electrons from the electron gun 505 to bombard the electron bombardment target 510 at the first position 501 or the second position 502. The electron gun 505 may be configured to generate an electron beam and then deflect or steer the generated electron beam to the electron bombardment target 510. The electron bombardment target 510 may be a plate comprising a material of a high atomic weight such as tungsten (W).

In one embodiment, when a bombarding electron from the electron gun 505 hits the electron bombardment target 510 at the first position 501 or the second position 502, there may be 3 possibilities. The first possibility is that the bombarding electron interacts with the nucleus of an atom of the electron bombardment target 510 and loses energy via the emission of a particle of radiation from the bombardment position. This process is usually referred to as the Bremsstrahlung process.

The second possibility is that the bombarding electron knocks an orbital electron out of an inner shell of an atom of the electron bombardment target 510. In response, another electron from an outer shell of the atom fills the resulting vacancy in the inner shell and thereby releases energy via the emission of a particle of radiation from the bombardment target 510. This process is usually referred to as the X-ray fluorescence process (or the characteristic X-ray emission process). The third possibility is that the bombarding electron causes the target 510 to heat up without causing any X-ray emission.

The electron gun 505 may be configured to generate electrons with high energy so that when these generated electrons bombard the electron bombardment target 510, these bombarding electrons have enough energy to cause the emission of particles of radiation (i.e., X-ray photons) from the electron bombardment target 510 according to either the first or second possibility mentioned above or both.

According to an embodiment, when the electron gun 505 shoots electrons of sufficiently high energy to the electron bombardment target 510 at different positions 501 or 502, these bombarding electrons cause the emission of particles of radiation from the different positions on the radiation source 500 towards a scene 50 and the image sensor 9000.

In the example shown in FIG. 4A, the image sensor 9000 and the radiation source 500 may remain stationary relative to the scene 50. In one embodiment, the radiation source 500 is configured to cause electrons from the electron gun 505 to bombard the electron bombardment target 510 at the first position 501 or the second position 502, for example, by translating the electron bombardment target 510 along a first direction 551 as shown in FIG. 4A or directing electrons from the electron gun 505.

In the example shown in FIG. 4A, after particles of radiation emitted from the first positions 501 on the radiation source 500 toward the scene 50, a first partial image 1010 of portions of the scene 50 is captured by the image sensor 9000 using the particles of radiation from only the first position 501, according to an embodiment. A second partial image 1020 of portions of the scene 50 may be captured by the image sensor 9000 using the particles of radiation emitted from only the second position 502.

In the example shown in FIG. 4B, the electron bombardment target 510 may move from a first position 501 relative to the electron gun 505 to a second position 502 by tilting, or both tilting and translating.

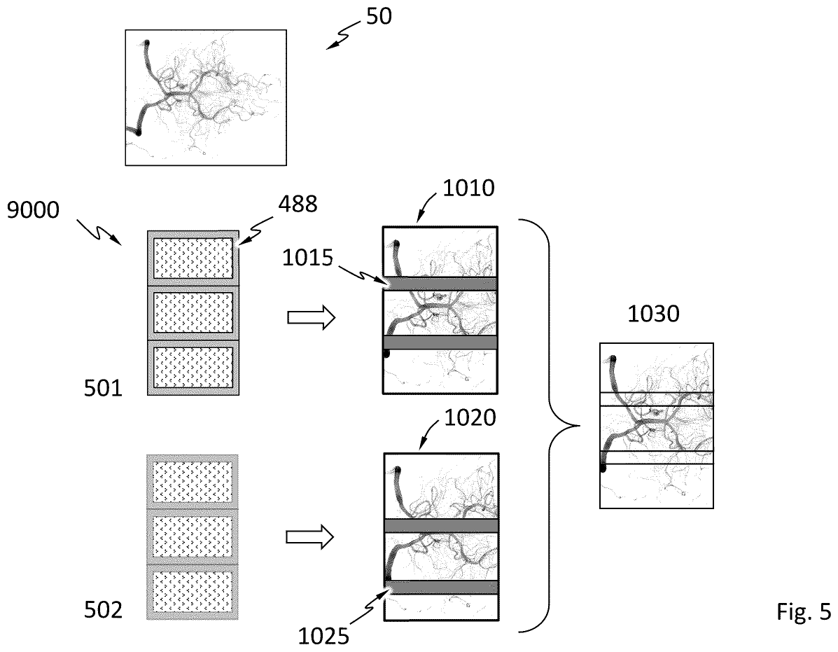

FIG. 5 schematically shows the image sensor 9000 capturing a plurality of partial images of portions of the scene 50, according to an embodiment. In the example shown in FIG. 5, the image sensor 9000 may capture partial images 1010 and 1020 of the portions of the scene 50 using the particles of radiation from only the first position 501 of the radiation source 500 and the second position 502 of the radiation source 500, respectively. The image sensor 9000 may stitch the partial images 1010, 1020 to form an image 1030 of the entire scene 50. The dead zones 488 of the image sensor 9000 may cause void in the partial images 1010 and 1020. In the example shown in FIG. 5, a void 1015 in the partial image 1010 is formed by the particles of radiation from only the first position 501 of the source 500 falling on the dead zone 488 of the image sensor 9000; a void 1025 in the partial image 1020 is formed by the particles of radiation from only the second position 502 of the source 500 falling on the dead zone 488 of the image sensor 9000, according to one embodiment. Since the dead zones 488 do not detect incident particles of radiation, the voids 1015 and 1025 captured in the partial images 1010 and 1020 respectively do not include image data of portions of the scene 50 and may be shown as blank areas as in the examples in FIG. 5. In one embodiment, each point in the scene 50 falls on the dead zone 488 of the image sensor 9000 no more than once when the image sensor 9000 is capturing partial images of the scene 50 using particles of radiation emitting from different positions of the source 500, respectively. According to an embodiment, each point in the scene 50 is captured in at least two partial images formed by particle of radiation from different positions on the radiation source 500 by the image sensor 9000. Therefore, the image 1030 of entire scene may be formed by combining the partial images (i.e., 1010, 1020, etc.) captured by the image sensor 9000 by using the particles of radiation emitted from the plurality of positions of the radiation source 500 without losing any portions of the scene 50 due to the voids caused by the dead zone 488 in the partial images.

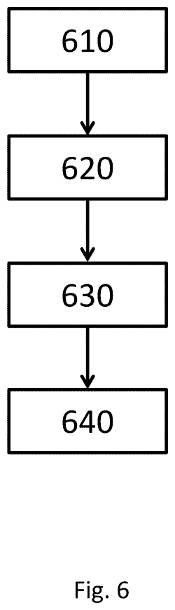

FIG. 6 schematically shows a flowchart for a method of operating the imaging system of FIGS. 4A and 4B, according to an embodiment. In procedure 610, an object is placed in the imaging system, e.g., for blood vessel digital subtraction angiography (DSA) imaging. The object may be a portion of a human body. In procedure 620, a first partial image of the object is captured by an image sensor using particles of radiation emitted from only a first position on a radiation source. The radiation source is configured to emit particles of radiation by bombarding an electron bombardment target at the first position using electrons from an electron gun. The electron bombardment target comprises tungsten, according to an embodiment. In procedure 630, a second partial image of the object is captured by the image sensor using particles of radiation emitted from only a second position of the radiation source. The radiation source is configured to emit particles of radiation from the second position by moving the electron bombardment target relative to the electron gun. Finally, in procedure 640, the first partial image and the second partial image may be stitched to form a full image of the entire object.

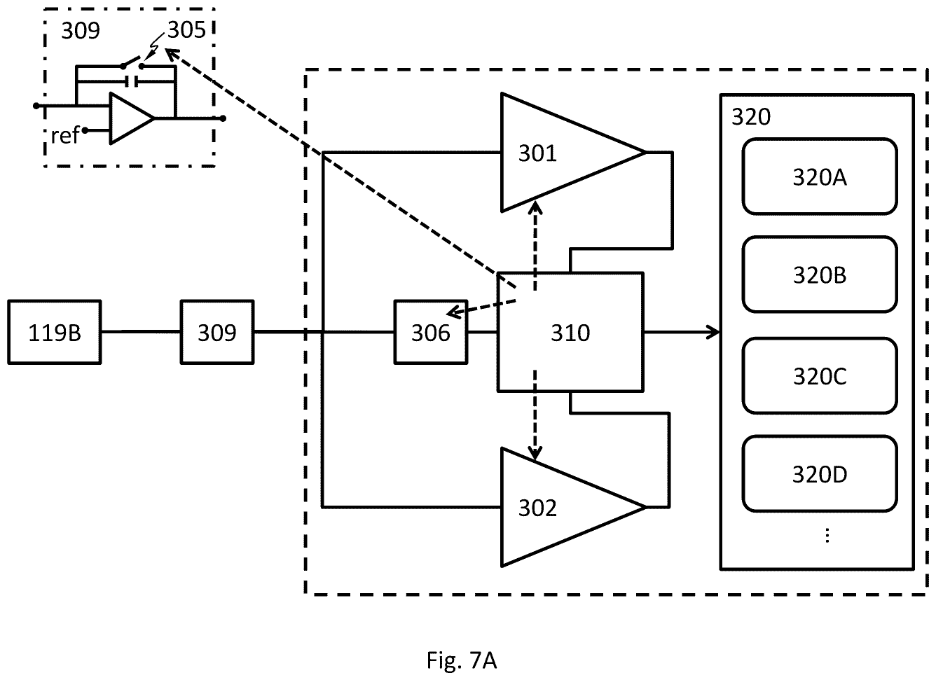

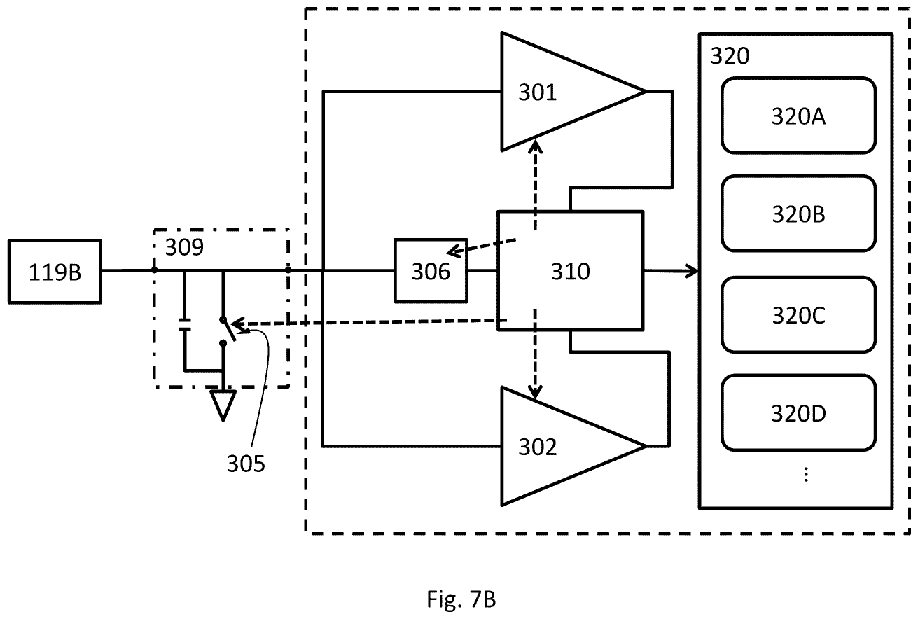

FIG. 7A and FIG. 7B each show a component diagram of the electronic system 121 of the radiation detector 100, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or calculate the voltage by integrating an electric current flowing through the electrical contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident particle of radiation may generate on the electric contact 119B. The maximum voltage may depend on the energy of the incident particle of radiation, the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” Ix′ of a real number x is the non-negative value of x without regard to its sign. Namely,

$$\vert x \vert = \{ \begin{matrix}x , & if x \geq 0 \\ - x , & if x \leq 0\end{matrix} .$$

The second threshold may be 200%-300% of the first threshold. The second threshold may be at least 50% of the maximum voltage one incident particle of radiation may generate on the electric contact 119B. For example, the second threshold may be 100 mV, 150 mV, 200 mV, 250 mV or 300 mV. The second voltage comparator 302 and the first voltage comparator 301 may be the same component. Namely, the system 121 may have one voltage comparator that can compare a voltage with two different thresholds at different times.

The first voltage comparator 301 or the second voltage comparator 302 may comprise one or more op-amps or any other suitable circuitry. The first voltage comparator 301 or the second voltage comparator 302 may have a high speed to allow the electronic system 121 to operate under a high flux of incident particles of radiation. However, having a high speed is often at the cost of power consumption.

The counter 320 is configured to register a number of particles of radiation incident on the radiation absorption layer 110 comprising pixels 150. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In one embodiment, the controller 310 is configured to activate the second voltage comparator at the beginning or expiration of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause at least one of the number registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In one embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In one embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 may include a capacitor directly connected to the electric contact 119B.

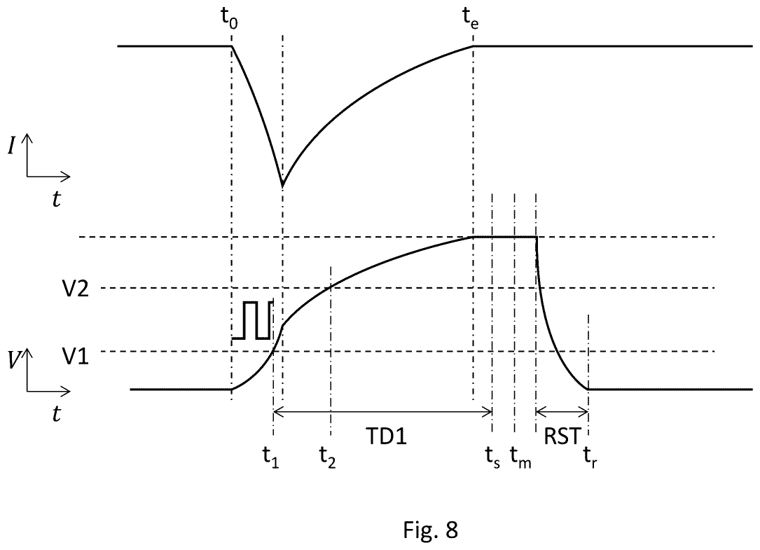

FIG. 8 schematically shows a temporal change of the electric current flowing through the electric contact 119B (upper curve) caused by charge carriers generated by a particle of radiation incident on the pixel 150 encompassing the electric contact 119B, and a corresponding temporal change of the voltage of the electric contact 119B (lower curve). The voltage may be an integral of the electric current with respect to time. At time to, the particle of radiation hits pixel 150, charge carriers start being generated in the pixel 150, electric current starts to flow through the electric contact 119B, and the absolute value of the voltage of the electric contact 119E3 starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize. The voltage stabilizes at time tₑ, when all charge carriers generated by the particle of radiation drift out of the radiation absorption layer 110. At time tₛ, the time delay TD1 expires. At or after time tₑ, the controller 310 causes the voltmeter 306 to digitize the voltage and determines which bin the energy of the particle of radiation falls in. The controller 310 then causes the number registered by the counter 320 corresponding to the bin to increase by one. In the example of FIG. 8, time tₛ is after time tₑ; namely TD1 expires after all charge carriers generated by the particle of radiation drift out of the radiation absorption layer 110. If time tₑ cannot be easily measured, TD1 can be empirically chosen to allow sufficient time to collect essentially all charge carriers generated by a particle of radiation but not too long to risk have another incident particle of radiation. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the difference between the voltage and the contribution to the voltage by the dark current is thus substantially zero at tₑ. The controller 310 may be configured to deactivate the second voltage comparator 302 at expiration of TD1 or at t₂, or any time in between.

The voltage at time tₑ is proportional to the amount of charge carriers generated by the particle of radiation, which relates to the energy of the particle of radiation. The controller 310 may be configured to determine the energy of the particle of radiation, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the electronic system 121 is ready to detect another incident particle of radiation. lithe first voltage comparator 301 has been deactivated, the controller 310 can activate it at any time before RST expires. If the controller 310 has been deactivated, it may be activated before RST expires.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
emitting particles of radiation from a first position on a radiation source toward a scene;
capturing a first partial image of the scene by an image sensor using the particles of radiation from only the first position;
emitting the particles of radiation from a second position on the radiation source toward the scene, the second position being different from the first position relative to the scene;
capturing a second partial image of the scene by the image sensor using the particles of radiation from only the second position;
forming an image of the scene by stitching the partial images;
wherein the image sensor comprises radiation detectors arranged in strips;
wherein the image sensor has dead zones among the strips;
wherein a portion of the scene in the first partial image is formed by the particles of radiation from only the first position falling on the dead zones of the image sensor;
wherein the portion of the scene in the second partial image is formed by the particles of radiation from only the second position falls on active areas of the image sensor.

2. The method of claim 1, wherein the image sensor remains stationary relative to the scene.

3. The method of claim 1, wherein each point in the scene is captured in at least two partial images formed by particle of radiation from different positions on the radiation source.

4. The method of claim 1, wherein the radiation source is stationary relative to the scene.

5. The method of claim 1, wherein the radiation source comprises an electron gun and electron bombardment targets.

6. The method of claim 5, wherein the radiation source is configured to cause electrons from the electron gun to bombard the electron bombardment target at the first position or the second position.

7. The method of claim 5, wherein the radiation source is configured to cause electrons from the electron gun to bombard the electron bombardment target at the first position or the second position by moving the electron bombardment target relative to the electron gun.

8. The method of claim 5, wherein the electron bombardment target is configured to tilt, translate, or both tilt and translate.

9. The method of claim 5, wherein the electron gun is configured to generate an electron beam and then deflect the electron beam.

10. The method of claim 5, wherein the electron bombardment target comprises tungsten.

11. The method of claim 1, wherein the image sensor comprises a plurality of pixels; wherein the pixels are configured to count numbers of the particles of radiation incident on the pixels, within a period of time.

12. The method of claim 1, wherein the particles of radiation are X-ray photons.

13. The method of claim 1, wherein the image sensor further comprises a plurality of radiation detectors that comprise:
a radiation absorption layer comprising an electric contact;
a first voltage comparator configured to compare a voltage of the electric contact to a first threshold;
a second voltage comparator configured to compare the voltage to a second threshold;
a counter configured to register a number of particles of radiation incident on the radiation absorption layer;
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause at least one of the numbers of particles to increase by one, when the second voltage comparator determines that an absolute value of the voltage equals or exceeds an absolute value of the second threshold.

14. The method of claim 13, wherein the image sensor further comprises an integrator electrically connected to the electric contact, wherein the integrator is configured to collect charge carriers from the electric contact.

15. The method of claim 13, wherein the controller is configured to activate the second voltage comparator at a beginning or expiration of the time delay.

16. The method of claim 13, wherein the controller is configured to connect the electric contact to an electrical ground.

17. The method of claim 13, wherein a rate of change of the voltage is substantially zero at expiration of the time delay.

18. The method of claim 13, wherein the radiation absorption layer comprises a diode.

19. The method of claim 13, wherein the radiation absorption layer comprises single-crystalline silicon.

20. The method of claim 13, wherein the radiation detector does not comprise a scintillator.
